(19) United States
(12) Patent Application Publication
(10) Pub. No.: US 2009/0036857 A1
(43) Pub. Date: Feb. 5, 2009

(54) Disposable feminine underwear

(76) Inventor: Denise Sherrod, Newport News, VA (US)

Correspondence Address: Bradley D. Goldizen, One Columbus Center, Ste. 665, Virginia Beach, VA, 23462, US

(21) Appl. No.: 11/888,724

(22) Filed: Aug. 2, 2007

(51) Int. Cl. A61F 13/15

(52) U.S. Cl. 604/385.3; 604/385.01; 604/393; 604/396

(57) ABSTRACT

A disposable undergarment for use by a female during a menstruation period includes a soft, stretchable, breathable material having a waist opening and two extended leg openings. The disposable undergarment extends from the waist opening arranged substantially at a navel region of the female to mid-thigh. The extended leg openings include an elastic edge around each open end for preventing leakage of menstrual flow. An absorbent region extends from the back region of the undergarment through the crotch region and upward to midway of the front region.

There are no related patent applications.

This application did not receive any federal research and/or development funding.

### TECHNICAL FIELD

The present invention generally relates to a disposable undergarment that may be worn during a woman's menstruation period. More specifically, the invention is disposable underwear comprising lightweight, breathable, stretchable material that includes a novel integrally formed absorbent region arranged in the crotch, front, rear and inside leg regions of the undergarment. The disposable underwear secures the absorbent regions in the most beneficial relationship to the woman's body and is useful when sleeping.

### BACKGROUND OF THE INVENTION

Menstruation is a phase of the menstrual cycle in which the uterine lining is shed. Typically, menstrual cycles occur in female humans once a month. During this period of time, the female may retain excessive water to become bloated and experience bleeding. This bleeding may become heavy on some days causing her to experience a heavy menstrual flow.

There are several known disposable products that aid women during her menstrual period. Some of these include sanitary napkins or pads that are rectangular pieces of material worn in their underwear. Many of these include “wings” or side pieces that fold around the crotch region of the panties. An adhesive backing on the wings holds the pads in place. Other types of pads are placed within the inner labia to absorb menstrual flow. Other known aids include tampons which are disposable wads of material that are inserted into the vagina to absorb menstrual flow.

The inventor has discovered several prior art devices directed towards aiding women. U.S. Pat. No. 6,387,084 to VanGompel et al. discloses a sanitary napkin with garment attachment panels. The sanitary napkin has longitudinal side edges that each include a garment attachment panel on the underside thereof. The garment attachment panels confine the undergarment crotch portion under the absorbent.

U.S. Pat. No. 7,128,732 to Jackson discloses an undergarment protection article and method for providing side leakage protection for an undergarment and absorbent article positioning guidance for proper placement on the undergarment. A pair of preformed cuffs are in direct contact with the crotch region of the undergarment to secure the absorbent article thereto.

U.S. Pat. No. 6,899,701 to Carstens et al. discloses a highly efficient absorbent article for use with menstrual pant. The absorbent articles preferably maintain contact with at least a portion of the wearer's labia and are either flat or cup-shaped.

U.S. Pat. No. 6,530,091 to Takai et al. discloses a means for attachment of absorbent article for body exudates to an undergarment. A support is disposed between a sanitary napkin and an inner surface of the crotch region of the undergarment. The support is formed by a first sheet and a second sheet overlapping the first sheet.

U.S. Pat. No. 6,475,202 to Hirsch discloses flexible and breathable absorbent articles and their fixation to undergarments. The articles have an air permeable or breathable back sheet.

All of the aforementioned devices are arranged to only cover a crotch region of a panty. If the devices fail to maintain contact with the crotch region, then leakage may occur. Moreover, they are neither designed for comfort nor to prevent leakage during heavy periods of menstrual flow. If any of the prior art devices are worn to bed, the wearer may experience leakage when rolling over during sleep or when readjusting her posture or sleeping position.

Other known absorbent articles include disposable diapers that have been used for many years. However, these are bulky and uncomfortable to wear.

### SUMMARY OF THE INVENTION

The present invention is a lightweight, disposable undergarment having a unique pad arranged therein. The undergarment is comfortable enough to wear to bed at night. It is used by women when they have a heavy menstrual flow and is designed to be worn by women during heavy flow nights.

The disposable underwear includes extended leg openings that are arranged mid-thigh when worn. A pad, or a portion of a larger pad, is arranged along an inner side of each extended leg opening.

The disposable underwear includes a waist opening that encircles the waist of the wearer substantially around the navel area. A pad or a plurality of pads extends from substantially midway between the navel area of the women's body downward to the crotch region. The pad extends through the crotch region and upward to substantially to the waist opening on the backside of the underwear and the crotch regions. The pad further extends downward from either side of the crotch region and along an interior side of the extended leg opening. The extended leg openings preferably extend to mid-thigh on the wearer to prevent spillage or leakage of menstrual flow. This provides more comfort and security while resting or sleeping. It should be noted that the pad in the rear portion of the underwear extending between the crotch region and waist opening is wider than that in the front as shown in FIGS. 1A and 1B.

The disposable underwear may include elasticized leg openings. A strip of elastic surrounds the waist opening at a top end of the underwear. The inside includes an absorbent region that extends from the back through the crotch to midway of the front and along the inside of the leg openings. The leg openings may include an inseam. The arrangement of the pad prevents leakage in any position or posture that the wearer may assume. The pad may be formed from plastics including, but not limited to, polyethylene, polypropylene, polystyrene, polyurethanes, and polyvinyl chloride, or any other thermoset materials. Preferably, the undergarment includes an exterior cloth-like material that is hydrophobic or waterproof arranged above a region that covers the pad. Otherwise an exterior side of the pad that is in contact with the interior side of the material of the undergarment may be hydrophobic. Either may comprise a sheet of polyethylene material. The pad may comprise cellulose pulp or a mixture of super absorbent material.

It is an object of the invention to provide disposable underwear that comfortably stretches to accommodate water retention. The disposable underwear includes a pad, or a plurality of pads that extend in the front, crotch, back and inside leg regions to prevent leakage.

It is another object of the invention to create a disposable undergarment that may be worn during a woman's menstruation period and is comprised of a lightweight, breathable, stretchable material that includes an integrally formed absorbent region arranged in the crotch, front, rear and inside leg regions of the undergarment.

The above and further objects, details and advantages of the invention will become apparent from the following detailed description, when read in conjunction with the accompanying drawings.

### BRIEF DESCRIPTION OF THE DRAWINGS

FIG. 1A is a front view of the present invention. FIG. 1B is a back view.

FIG. 2A is a cross section view of a first embodiment of the invention and taken from the centerline shown in FIGS. 1A and 1B. FIG. 2B is a cross section view of a second embodiment of the invention taken from the centerline.

FIG. 3 is an overhead view of the pad when disassembled from the underwear.

FIG. 4 is a prior art underwear.

### DETAILED DESCRIPTION OF THE INVENTION

The embodiments of the invention and the various features and advantageous details thereof are more fully explained with reference to the non-limiting embodiments and examples that are described and/or illustrated in the accompanying drawings and set forth in the following description. It should be noted that the features illustrated in the drawings are not necessarily drawn to scale, and the features of one embodiment may be employed with the other embodiments as the skilled artisan recognizes, even if not explicitly stated herein. Descriptions of well-known components and techniques may be omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the appended claims. Moreover, it is noted that like reference numerals represent similar parts throughout the several views of the drawings.

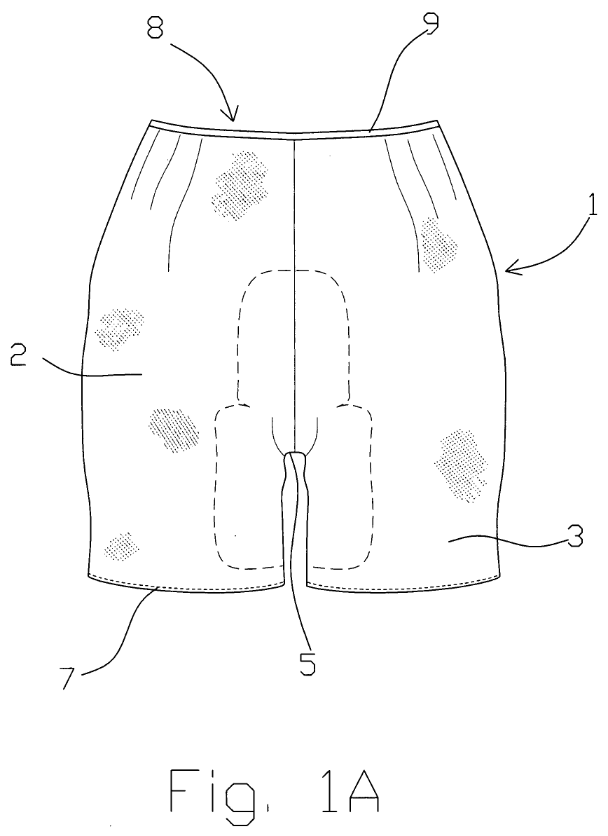

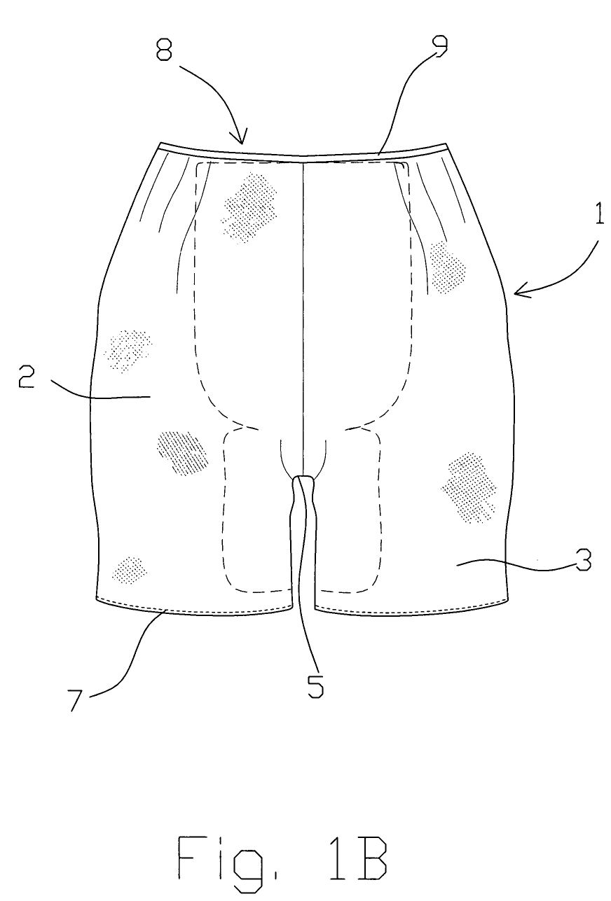

FIGS. 1A and 1B show the disposable underwear 1 that comprises material 2 and includes a pad 10 or a plurality of pads shown in phantom. The underwear 1 includes two extended leg openings 3 having stitched inseams 7 arranged at a lower end thereof. A waist opening 8 is provided at a top of the underwear 1 and includes an elasticized lip 9.

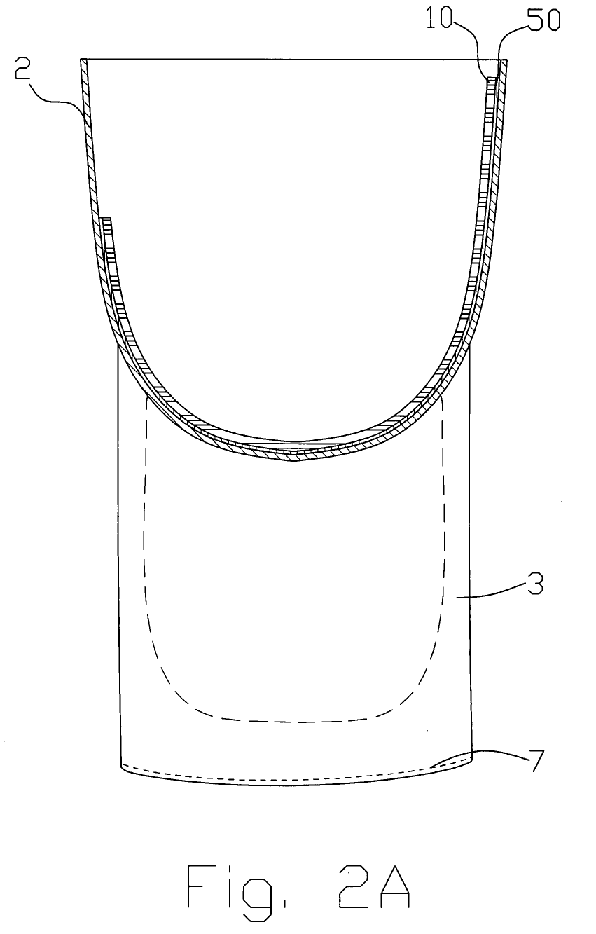

FIG. 2A shows a cross section view of the underwear 1 and pad 10 taken along the centerline shown in FIGS. 1A and 1B. A central portion 11 of the pad 10 is integrally arranged within the crotch of the underwear. As shown, the respective leg pads 12, 13 extend down and inside of the extended leg opening 3. The front pad 14 and rear pad 15 extend from the central portion 11 and upwards toward the waist opening 8.

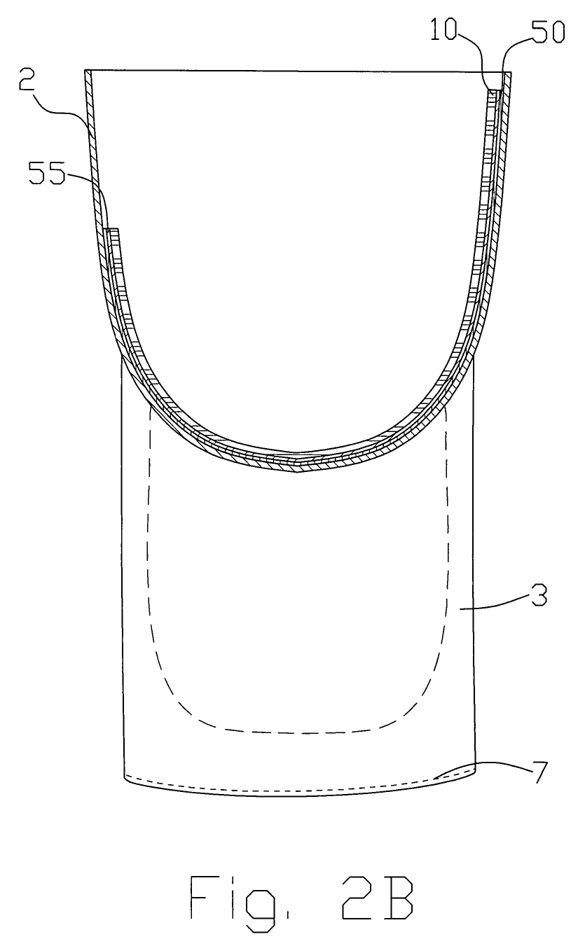

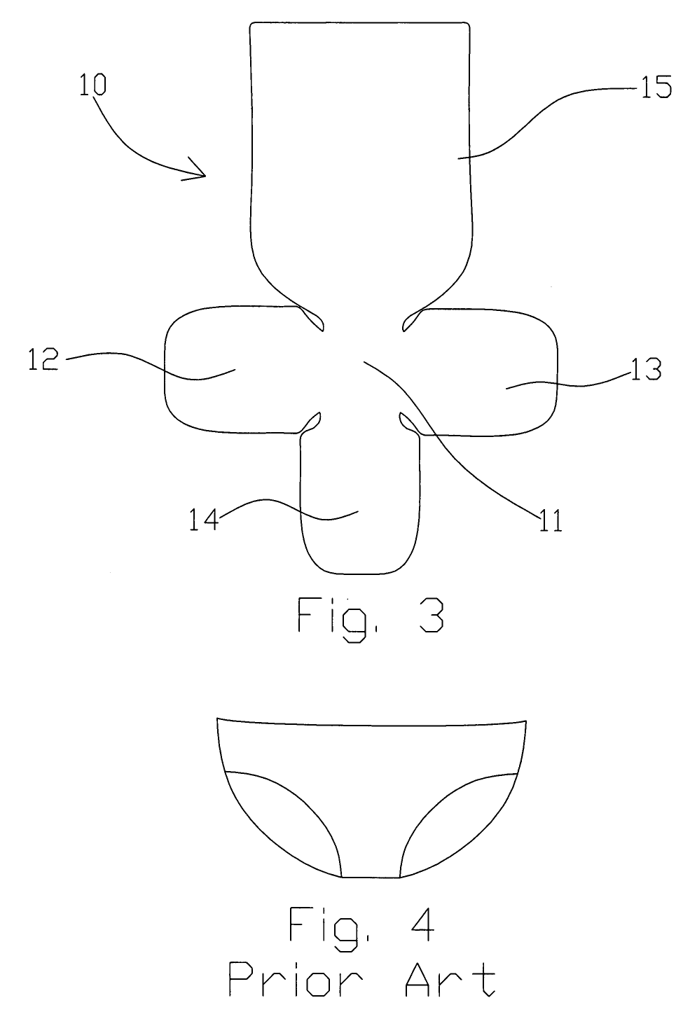

The pad 10 extends upward in the front and rear regions of the underwear 1 and downward on an interior side of the extended leg openings 3 that terminate in the stitched inseams 7. In the preferred embodiment, the underwear comprises lightweight laced cotton fabric which easily stretches to accommodate a particular shape of a women's body. The cotton fabric is woven to create a stretchable, breathable material that comfortably conforms to the body of the wearer. In one embodiment, the underwear includes a single ply of stretchable, breathable material having a pad formed integrally therewith. The pad is clover-leaf in shape when shown lying flat as in FIG. 3. An adhesive 50 secures the pad 10 to the inner side of the fabric 2. In FIG. 2B, an additional layer of hydrophobic material 55 is arranged between the pad and the adhesive to prevent any moisture from causing separation of the adhesive between the pad 10 and material 2 or leakage through the material 2 to an exterior side thereof.

FIG. 3 is an overhead view of a pad 10 including an absorptive material that may comprise one or more of the following materials including cellulose material, plastics including, but not limited to, polyethylene, polypropylene, polystyrene, and polyurethanes. As the padding becomes soaked it will expand away from the vaginal area. Preferably, the back side of the pad 10 that is in contact with the material 2 is breathable and comprises a hydrophobic material. As shown in FIG. 3, the front pad is substantially the same size as the left leg pad 12 and right leg pad 13, as shown. However, the back pad 15 is substantially larger than any of the other pads. As shown, the pads are preferably cut with a removed area 25 there between to allow for them to be correctly position within the material 2. Adhesive may be disposed between material 2 and pad 10 to fasten them together. Each of the pads 12-15 extend from the central region 11.

FIG. 4 is a view of a prior art panty. In this instance, the prior art panty is reusable and includes leg openings arranged on either side of the crotch.

While the invention has been described with respect to preferred embodiments, it is intended that all matter contained in the above description or shown in the accompanying drawings shall be interpreted as illustrative and not in limiting sense. From the above disclosure of the general principles of the present invention and the preceding detailed description, those skilled in the art will readily comprehend the various modifications to which the present invention is susceptible. Therefore, the scope of the invention should be limited only by the following claims and equivalents thereof.

I claim:

1. Disposable underwear comprising:
lightweight breathable material having an elasticized waist opening, a crotch region, a front side, a back side and two extended leg openings having an inner side, said lightweight breathable material further comprising an outer side and an inner side; and,
a pad of absorbent material having a central region arranged in the crotch region of the light weight breathable material and extending upwards on an interior of the front side and extending upwards on an interior of the back side of lightweight breathable material, said pad of absorbent material extending downwards at least along a portion of the inner side of the extended leg openings.

2. The disposable underwear of claim 1 further comprising:
a layer of hydrophobic material arranged between the pad of absorbent material and the lightweight breathable material.

3. The disposable underwear of claim 1 further comprising:
inseams formed at an end of each extended leg opening.

4. The disposable underwear of claim 1 wherein said pad comprises one or more selected from a group consisting of polyethylene, polypropylene, polystyrene, polyurethanes, and polyvinyl chloride, or thermoset materials.

5. The disposable underwear of claim 1 wherein said lightweight breathable material includes hydrophobic material.

6. The disposable underwear of claim 1 wherein said underwear comprises polyethylene.

7. The disposable underwear of claim 1 wherein said pad comprises cellulose pulp.

8. A disposable undergarment comprising:
formed lightweight breathable material having an elasticized waist opening, a crotch region, a front side, a back side and two extended leg openings each having an inner side, said lightweight breathable material further comprising an outer side and an inner side;
a pad of absorbent material having a central region arranged in the crotch region of the light weight breathable material and extending upwards on an interior of the front side and extending upwards on an interior of the back side of lightweight breathable material, said pad of absorbent material extending downwards at least along a portion of the inner side of the extended leg openings and,
wherein said lightweight breathable material includes hydrophobic material arranged at least in an area in contact with said pad.

9. The disposable undergarment of claim 8 wherein said pad comprises one or more selected from a group consisting of polyethylene, polypropylene, polystyrene, polyurethanes, and polyvinyl chloride, or thermoset materials.

10. The disposable undergarment of claim 8 wherein said undergarment comprises polyethylene.

11. The disposable undergarment of claim 8 wherein said pad comprises cellulose pulp.

12. A disposable undergarment comprising:
formed lightweight breathable material having an elasticized waist opening, a crotch region, a front side, a back side and two extended leg openings each having an inner side, said lightweight breathable material further comprising an outer side and an inner side;
a pad of absorbent material having a central region arranged in the crotch region of the light weight breathable material and extending upwards on an interior of the front side and extending upwards on an interior of the back side of lightweight breathable material, said pad of absorbent material extending downwards at least along a portion of the inner side of the extended leg openings and,
wherein said lightweight breathable material includes hydrophobic material arranged at least in an area in contact with said pad and,
wherein said pad comprises one or more selected from a group consisting of polyethylene, polypropylene, polystyrene, polyurethanes, and polyvinyl chloride, or thermoset materials.

13. The disposable undergarment of claim 12 wherein said undergarment comprises polyethylene.

14. The disposable undergarment of claim 12 wherein said pad comprises cellulose pulp.
